(19) United States
(12) Patent Application Publication
(10) Pub. No.: US 2007/0106109 A1
(43) Pub. Date: May 10, 2007

(54) Device for Stone Therapy and Sexual Stimulation

(76) Inventor: Van Bich Ly, McLean, VA (US)

Correspondence Address: ROSSI, KIMMS & McDOWELL LLP., P.O. BOX 826, ASHBURN, VA, 20146-0826, US

(21) Appl. No.: 11/164,099

(22) Filed: Nov. 10, 2005

(51) Int. Cl. A61F 5/00

(52) U.S. Cl. 600/38

(57) ABSTRACT

A rock, stone, semi-precious crystal, or mineral stone material(s) shaped and formed to be used to provide and improve sexual stimulation to a user. The rock, stone, semi-precious crystal, or mineral stone sexual stimulation tool is of a sufficient weight and size to reproduce the weight and sensation of a penis. In one embodiment, the present invention comprises a smooth oblong-shaped rock of various lengths and diameter. In another embodiment, the invention is shaped like a dildo or other phallic device. In another embodiment, the invention has an internal chamber for the addition of vibration-producing device or a heating element, in which a controlled trigger permits the user to activate continuous vibration or to retain a particular temperature.

### FIELD OF THE INVENTION

The present invention relates to devices for stone therapy, and more particularly, to a device that incorporates stone therapy with sexual stimulation.

### BACKGROUND OF THE INVENTION

Stone therapy has become more and more popular as a form of massage therapy. In general, stone therapy involves placing stones on various points of a person's body. The weight of the stones is therapeutic, and the heating and cooling of the stones before contacting them with the person's body provides a refreshing way to blend the body, mind, and soul to achieve a state of relaxation. Various stones can be used in stone therapy, and various textures, shapes, and placements help to relax the person.

For example, in stone therapy, stones are generally not placed on bones and joints. Stones are placed anywhere else though, and can even placed between the person's toes. Essentially, the person feels the varying hot and cold temperatures of the stones and achieves a different mental state then normally possible because of the pressures and temperatures transmitted by the stones. Some stones transmit heat faster than other stones, and thus, how quickly stones heat or cool a particular place on a person's body can be controlled depending upon the type of stone employed. Similarly, the temperature of stones can vary extremely from the person's normal body temperature, or the temperature of the stones can only be slightly different than the person's normal body temperature.

Stone therapy is chronicled at http://www stonetherpyschool.com/about stones.cfm, wherein therapy generally involves 46 basalt heated stones and six marble stones employed for 60-75 minutes, and up to two hours. According to this web site, the basalt stones are heated to the temperature of 125 to 135 degrees Celsius in an 18-quart roaster oven. The marble stones are cold but not frozen. It is noted that some of the basalt stones are used on the person's back while other basalt stones are used on the person's palms. Essentially, as described on this web site, the stones give their heat and electromagnetic energy to the person's body to feed, relax, and bring sense of peace to the person's body. The polycrystalline structure of basalt stones allows the waves of heat to slowly emanate from the basalt stones, and the warmth attracts blood in the person's body to the surface of the person's skin. The cold marble stones provide coolness penetrating deeply and quickly into the person's skin because of marble's chemical composition and monocrystalline structure. The person's body feels fresh and clean, and the person's body feels as if a fresh mountain breeze has just entered it. The coolness of the marble stones is especially well received after right after the hot basalt stones have been employed.

While stone therapy is becoming more mainstream, it is restricted to therapy, and not sexual stimulation. It is desirable to incorporate the beneficial and enjoyable elements of stone therapy in some form that provides sexual stimulation heightened by the stone therapy. Thus, the user would not only feel the stone therapy on parts of the body that are asexual, but the user would feel stone therapy on sexual parts of the body.

Sexual toys and devices are far from new, and some rather unique sexual devices can be found at http://www.mi-su.com/homepage.asp. This web page showcases sex toys of sculptural shapes and certain exotic materials such as solid titanium, rose quartz, and obsidian. The sex toys are adorned with diamonds, sapphires, and other precious and semi-precious stones. This web page notes that “the range is both decorative and fun.” Thus, while materials beyond the typical sex toy materials have been chosen for constructing the sex toys of this web site, there is no recognition of how such materials can possibly be employed in a functional manner aside from the normal functional properties of sex toys.

Thus, there still remains a need to bring stone therapy to the sexual arena, such that the energy and desires associated with sex toys can be mated to the sensations of stone therapy.

### SUMMARY OF THE INVENTION

The present invention employs the traditional materials used in stone therapy, and/or similar materials, in a heated and cooled fashion to deliver stone therapy to the genitals. While the waves of heat and refreshing coolness common in stone therapy are beneficial, new and improved sensations and satisfaction can be achieved when stone therapy is applied to the genital area.

According to the present invention, it is an object of the present invention to provide a stone, dildo or phallic in shape, that is made from natural and organic material(s). The present invention resolves the market limitations for those who take special care in consuming exclusively organic and natural products.

While there are a host of sexual products made from synthetic materials, the present invention has no synthetic materials, and thus, the present invention is safe to be used in a wide range of temperatures that would normally cause synthetic materials to breakdown, crack, and discolor. Accordingly, it is an object of the present invention to allow the user a wider range of temperature zones with which to apply the sex toy.

Further, the present invention can be washed in the dishwasher at all temperatures. To use the present invention at an elevated temperature, it is as easy as immersing the present invention in a cup or bowl of water at the temperature that the user desires.

The present invention is a sexual aid device for providing sexual stimulation. Dildos and other phallic devices have been used for years by humans, either as a substitute to a sexual partner or to enhance the level of sexual excitement during sex between partners. In the past, the problems and limitations with respect to tools, methods, and satisfaction levels includes: the materials used, means of heat/cold retention, the amount of heat/cold for application, weight of the tool, and negative perception.

Dildos made of softer and “realistic” feeling dildos make the assumption that the female sexual anatomy, the vagina, is as sensitive as the skin on fingers or other parts of the body. In fact, the vagina can only sense what is significantly different in temperature, size, and force. Thus, to make a truly effective dildo, the dildo must be variable but controllable in terms of temperature, size, and force.

Conventional dildos have been created out of materials such as Pyrex, acrylic, and silicone; each of these materials come with their own respective limitations. Pyrex is glass, and thus, it breaks easily and carries little weight. Weight is desirable for a dildo though. Acrylic can be easily damaged by high heat, alcohol and other solvents, not to mention that Acrylic carries little weight and scratches easily. Silicone and jelly are rubber substances synthesized by humans, carry little weight, and tend to have an odor.

Conventional dildos made of Pyrex, acrylic, and silicone lack the natural property of heat and temperature transfer and retention. The assumed method of use of conventional dildos is either at room temperature or slightly warmed for comfort. Additionally, these materials may not be able to withstand the high heat, either for cleaning purposes, for the purpose of use, or when accidentally overheated.

The weight of the tool impacts on its ability to reproduce the sensation and weight of a male partner. It is not the actual weight of the penis that creates the sensation of weight and tension within the female partner. It is the weight and force of the male partner that creates the sensation of weight and tension within the female partner. Dildos made of Pyrex, acrylic, and silicone tends to be lightweight, and thus, do not provide a desired sensation.

The combination of limitations and problems contributes to the overall negative perception of dildos and other sexual aid device. Additionally, negative social perceptions and norms reduce the level of satisfaction in using dildos and other sexual aids/tools.

It is an object of the present invention to provide a more realistic and accurate sensation of the force and weight of an actual male partner. The present invention is not merely shaped to resemble the male genitals, but moreover, the present invention provides the weight necessary to help the user come closer to feeling the pressure delivered by the male genitals during sexual intercourse. The pressure, coupled with temperature changes in the present invention, provides a heretofore unfamiliar sensation.

It is an object of one feature of the present invention to change the social perception, and a user's perception, of human sexuality. The present invention is a positive contribution and a positive factor in human spirituality and thus, to human sexuality. Rather than merely acting as a sex toy, the present invention serves to meld therapy with sexual stimulation. Thus, sexuality is moved from something discrete into the realm of actual therapy.

The present invention is an improvement over the conventional dildos and current methods of sexual stimulation. The present invention, by expanding the concept of sexual stimulation to become sexual therapy, opens the normally stigmatized door of sex toys and moves them into an area of spiritual energy channeling, as similarly found with stone massage therapy.

### BRIEF DESCRIPTION OF THE DRAWINGS

FIG. 1 is a view of the plug removed from the main body of the present invention.

### DETAILED DESCRIPTION OF THE PREFERRED EMBODIMENTS

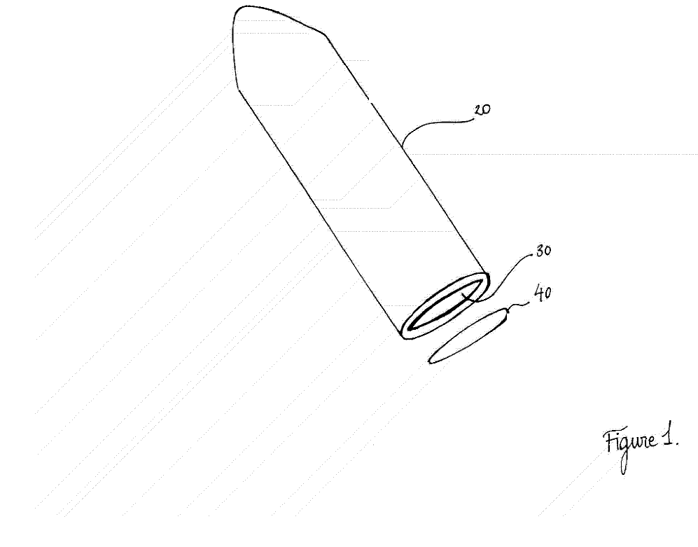

FIG. 1 shows the present invention. It is comprised of a main body (20), a plug (30) and a chamber (40). The chamber (40) is capable of housing a conventional vibrating mechanism, or in an alternative embodiment can be filled with a hot or cold substance to alter the temperature of the apparatus.

Sexual arousal typically brings blood to the aroused sexual anatomy. The invention at an elevated temperature becomes a catalyst for bringing blood to the sexual anatomy and increases the blood circulation. For a man, the increased blood circulation to the penis and the surrounding area can either help bring a man to a greater level of arousal, an increase in size and in hardness, or aid in the achievement of an orgasm. For a woman, the increased blood circulation to the vagina area increases the level of sexual excitement and aid in the achievement of an orgasm.

It is suggested the user start with a small temperature variation from the existing room temperature, when the user wants to use the present invention cold. The user should work gradually and slowly toward the extreme temperature of 32 degrees Fahrenheit. At each degree of change, the user should test the body comfort on different areas of the body, such as palm of hand, inner thigh, and outer parts of the genitals. It is not however, required nor advised to use the invention at this extreme temperature.

To use the present invention cold, the user should place it in the refrigerator before use or have it submerged in a bowl filled with ice and water. To ensure proper temperature for use, a temperature gauge should be submerged along with the invention. The amount of time required to chill the stone is dependent on its existing temperature and the amount of time to reach the desired temperature. However, it may be necessary keep the invention at that temperature point for at least 5 minutes to ensure that it will retain this temperature longer during use.

For a heated use, the invention can be warmed in a crock pot filled with water, as similarly used to heat hot stones for massage therapy, or submerged in a bowl filled with hot water. Again a temperature gauge should be submerged along with the present invention. It may be necessary keep the invention at that temperature point for at least 5 minutes to ensure that it will retain this temperature longer during use. It is suggested that the user start with a small temperature variation from the existing body temperature, when the user wants to use the present invention hot. The user should work gradually and slowly toward the extreme temperature of 140 degrees Fahrenheit. At each degree of change, the user should test their body comfort on different spots of the body, such as palm of hand, inner thigh, and outer parts of the genitals. It is not however, required nor advised to use the invention at this extreme temperature.

When the invention comes with a heating element, the user simply dials in the amount of heat desired. It is possible the invention may come with an “ON” and “OFF” control, when the desired temperature is reached and then, turn it to “OFF”. In an alternative embodiment of the present invention, the main body (20) houses a chamber (30), which can hold a heated or cooled substance. The chamber (30) is accessed through the plug (40). It is preferred that the plug of the present invention be made of the same material as the main body (20) of the present invention so that weight, temperature, and texture of the plug (40) does not disturb any sensation transmitted to the user received from the main body (20).

In an alternative embodiment of the present invention, the main body (20) houses a conventional vibrating mechanism, thus allowing the user to further use the apparatus as a vibrator.

Due to the lack of study and knowledge of human thermosensory, use of this invention at specific temperatures is not precise or scientifically known. It is however, based on human satisfaction levels based on his or her own judgment of what is sexually enhancing and seductive.

The invention needs to be “pre-seasoned” with hydrating oil, such as jojoba oil. The invention can be cleaned by bathing in warm organic soap or boiled in water with a sprinkle of natural sea salt. This can be followed by an oil treatment as described above. Cleaning is advised before and after each use. Bathing in nature, such as a babbling brook, streams, creeks, waterfalls, and oceans, is the most natural cleansing for the invention and is a treat for the user.

Energy is lost when the invention is used, and recharging periodically helps maintain its energy. To energize and recharge the invention, it should be placed in a safe, clean location that will expose it to the elements such as direct sunlight and wind; although, with the exception of such materials as marble, too much exposure to the sun may dry out the material.

Having illustrated the present invention, it should be understood that various adjustments and versions might be implemented without venturing away from the essence of the present invention. The present invention is not limited to the embodiments described above, and should be interpreted as any and all embodiments within the scope of the following claims.

What is claimed is:

1. An apparatus for sexual stimulation for a user, comprising:
a main body made of an organic material;
a plug made of organic material, in communication with said main body.

2. The apparatus of claim 1 wherein said main body is made of an organic material such as rock, semi-precious crystal, or mineral.

3. The apparatus of claim 1 wherein said plug is made of an organic material such as rock, semi-precious crystal, or mineral.

4. The apparatus of claim 1 wherein said main body and said plug are configured to be heated or cooled.

5. The apparatus of claim 1 wherein said main body is configured to house a vibrating mechanism.

6. The apparatus of claim 1 wherein said plug provides access to a vibrating mechanism housed within said main body.

7. A method for using a sexual stimulation apparatus, comprising: placing the sexual stimulation apparatus in direct sunlight before use.

8. The method of claim 7, further comprising: seasoning the sexual stimulation apparatus.

9. The method of claim 7, further comprising: altering the temperature of the sexual stimulation apparatus.

10. The method of claim 7, further comprising: cleansing the apparatus.

11. The method of claim 8, further comprising: altering the temperature of the sexual stimulation apparatus.

12. The method of claim 8, further comprising: cleansing the apparatus.

13. The method of claim 9, further comprising: cleansing the apparatus.

14. The method of claim 9 wherein said altering of the temperature of said sexual stimulation apparatus occurs in the range of the temperatures of 32 to 140 degrees Fahrenheit.

15. The method of claim 11 wherein said altering of the temperature of said sexual stimulation apparatus occurs in the range of the temperatures of 32 to 140 degrees Fahrenheit.

16. The apparatus of claim 6 wherein said plug provides access to a chamber capable of holding a heated or cooled substance within said main body.
